(19) United States
(12) Patent Application Publication
(10) Pub. No.: US 2017/0333318 A1
(43) Pub. Date: Nov. 23, 2017

(54) DEODORIZING ACTIVE INGREDIENT COMBINATION

(71) Applicant: Henkel AG & Co. KGaA, Duesseldorf (DE)

(72) Inventors: Thomas Doering, Dormagen (DE); Natascha Schevardo, Erkrath (DE)

(73) Assignee: Henkel AG & Co. KGaA, Duesseldorf (DE)

(21) Appl. No.: 15/598,179

(22) Filed: May 17, 2017

(30) Foreign Application Priority Data
May 23, 2016 (DE) 102016208859.9

(51) Int. Cl. A61K 8/41; A61K 8/34; A61K 8/06; A61Q 15/00; A61K 8/37

(52) U.S. Cl. CPC A61K 8/416; A61Q 15/00; A61K 8/37; A61K 8/06; A61K 8/34; A61K 2800/30

(57) ABSTRACT

A deodorizing cosmetic agent in the form of an aqueous emulsion, including:
at least one quaternary ammonium compound, and
at least one menthol ester,
wherein the deodorizing cosmetic agent does not include any aluminum-containing compounds;
use thereof, and methods using said agent.

### FIELD OF THE INVENTION

The present invention generally relates to cosmetic deodorant compositions having an improved effect, and methods using said compositions.

### BACKGROUND OF THE INVENTION

In many cultures, cleaning of and care for one's own body includes measures to counteract the emergence of excessive unpleasant-smelling body odor. The emergence of body odor is encouraged, inter alia, by perspiration, for which reason the cosmetic industry offers many body care agents that have been developed for use in areas of the body that have a high density of sweat glands, in particular, in the armpit area. These body care agents act as antiperspirants and/or deodorants.

Cosmetic antiperspirants of the prior art normally include at least one oil or fatty substance and odorant component or perfume and, in addition thereto, at least one antiperspirant salt. On one hand, the antiperspirant compounds used in these antiperspirants reduce the secretion of sweat from the body by temporarily narrowing and/or blocking the ducts of the sweat glands so that the amount of sweat can be reduced by about 20 to 60%. On the other hand, these antiperspirant compounds have an additional deodorizing effect due to the antimicrobial action thereof. Alkaline aluminum and aluminum-zirconium halides (which usually have been activated) are used as antiperspirant salts. It is also possible to use aluminum and aluminum-zirconium halides that have been stabilized with organic acids as complex ligands. People with sensitive skin may react with skin irritation to the antiperspirant salts. Aluminum-free antiperspirants and deodorants are disclosed, for example, in German patent applications DE102013225617 A1 and DE102013225620 A1.

Antiperspirants and deodorants may be prepared in the most varied forms of application, for example, as a powder, in the form of a stick, as an aerosol spray, liquid and gelatinous roll-on application, cream, gel, and as an impregnated flexible substrate (deodorant wipes).

Manufacturing the antiperspirants and deodorants in liquid form has advantages in relation to dosage and application, e.g., by means of an atomizer or spray. Low-viscous emulsion systems that are suitable for spraying, however, often do not have sufficient storage stability.

### BRIEF SUMMARY OF THE INVENTION

It has now surprisingly been found that the aforementioned industrial problems are solved by aluminum-free compositions that include at least one quaternary ammonium compound in combination with esters of menthol. The present invention provides:

A deodorizing cosmetic agent in the form of an aqueous emulsion. The deodorizing cosmetic agent includes at least one quaternary ammonium compound, and at least one menthol ester. The deodorizing cosmetic agent does not include any aluminum-containing compounds.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In this context, the present invention addresses the industrial problem of providing a deodorant having high effectiveness against body odor, favorable skin compatibility, and high storage stability and microbiological stability.

It has now surprisingly been found that the aforementioned industrial problems are solved by aluminum-free compositions that include at least one quaternary ammonium compound in combination with esters of menthol. The present invention provides:

A deodorizing cosmetic agent in the form of an aqueous emulsion, including:
at least one quaternary ammonium compound, and
at least one menthol ester,
   wherein the deodorizing cosmetic agent does not include any aluminum-containing compounds.

The deodorizing cosmetic agent according to point 1, wherein the agent includes, as the quaternary ammonium compound, at least one compound selected from the group consisting of behentrimonium chloride, cetrimonium bromide, cetrimonium chloride, laurtrimonium bromide, laurtrimonium chloride, steartrimonium bromide, steartrimonium chloride, benzethonium chloride, benzalkonium chloride, and mixtures thereof.

The deodorizing cosmetic agent according to any of the preceding points, wherein the agent includes benzalkonium chloride as the quaternary ammonium compound.

The deodorizing cosmetic agent according to any of the preceding points, wherein the quaternary ammonium compound is included, relative to the total weight of the agent, in an amount of 0.01 to 0.3 wt %, preferably 0.02 to 0.2 wt % and, in particular, 0.05 to 0.1 wt %.

The deodorizing cosmetic agent according to any of the preceding points, wherein the agent includes menthyl acetate as the menthol ester.

The deodorizing cosmetic agent according to any of the preceding points, wherein the agent includes the menthol ester, relative to the total weight of the agent, in an amount of 0.01 to 10 wt %, preferably 0.03 to 5.0 wt % and, in particular, 0.05 to 0.5 wt %.

The deodorizing cosmetic agent according to any of the preceding points, wherein the agent includes at least one bactericide from the group consisting of chlorhexidine, farnesol, triclosan, chloroxylenol, and phenoxyethanol, preferably phenoxyethanol.

The deodorizing cosmetic agent according to any of the preceding points, wherein the agent includes 0.1 to 2.0 wt %, preferably 0.2 to 1.6 wt % and, in particular, 0.5 to 1.4 wt % a bactericide, preferably phenoxyethanol.

The deodorizing cosmetic agent according to any of the preceding points, wherein the agent includes at least one oil that is liquid at 20° C. and 1,013 hPa from the group consisting of
esters of linear or branched saturated or unsaturated C₂₋₃₀ fatty alcohols with linear or branched saturated or unsaturated C₂₋₃₀ fatty acids,
addition products of ethylene oxide and/or propylene oxide with monohydric or polyhydric C₃₋₂₂ alkanols
dialkyl ethers, and
paraffins.

The deodorizing cosmetic agent according to any of the preceding points, wherein the agent includes at least one oil that is liquid at 20° C. and 1,013 hPa, relative to the total weight of the agent, in an amount of 0.1 to 15 wt %, preferably 0.2 to 10 wt % and, in particular 0.5 to 5.0 wt %.

The deodorizing cosmetic agent according to any of the preceding points, wherein the agent includes at least one non-ionic emulsifier from the group consisting of the addition products of 2 to 50 mol ethylene oxide with linear fatty alcohols having 12 to 18 carbon atoms and the addition products of 2 to 50 mol propylene oxide with linear fatty alcohols having 12 to 18 carbon atoms.

The deodorizing cosmetic agent according to point 11, wherein the agent includes the non-ionic emulsifier, relative to the total weight of the agent, in an amount of 3.0 to 12 wt %, preferably 4.0 to 10 wt % and, in particular, 5.0 to 8.0 wt %.

The deodorizing cosmetic agent according to any of the preceding points, wherein, relative to the total weight of the agent, ethanol is included in amounts below 5.0 wt %, preferably below 1.0 wt % and, in particular, below 0.1 wt %.

The deodorizing cosmetic agent according to any of the preceding points, wherein the agent is free of ethanol.

The deodorizing cosmetic agent according to any of the preceding points, wherein the agent exists as an oil-in-water emulsion.

The deodorizing cosmetic agent according to any of the preceding points, wherein, relative to the total weight of the agent, water is included in amounts of 75 to 96 wt %, preferably 80 to 96 wt % and, in particular, 85 to 94 wt %.

The deodorizing cosmetic agent according to any of the preceding points, wherein the agent is propellant-free.

A pump sprayer containing the deodorizing cosmetic agent according to any of the preceding points.

A roll-on applicator containing the deodorizing cosmetic agent according to any of the preceding points.

Use of the deodorizing cosmetic agent according to any of the preceding points to prevent and/or reduce body odor.

A non-therapeutic cosmetic method for preventing and/or reducing body odor, in which the cosmetic agent according to any of the preceding points is applied to the skin, in particular, to the skin of the armpits, and left on the skin for at least 1 hour, preferably for at least 2 hours, preferably for at least 4 hours, in particular, for at least 6 hours.

The cosmetic agents according to the present invention include at least one quaternary ammonium compound as the first essential component thereof. Cosmetic agents that include at least one compound selected from the group consisting of behentrimonium chloride, cetrimonium bromide, cetrimonium chloride, laurtrimonium bromide, laurtrimonium chloride, steartrimonium bromide, steartrimonium chloride, benzethonium chloride, and benzalkonium chloride as the quaternary ammonium compound have proven advantageous in terms of the industrial properties thereof, in particular, the storage stability and cosmetic effect thereof. The use of mixtures of the aforementioned quaternary ammonium compounds is, of course, also possible.

One quaternary ammonium compound that is especially preferred industrially is benzalkonium chloride.

The proportion by weight of the quaternary ammonium compounds to the total weight of the cosmetic agent is preferably 0.01 to 0.3 wt %, preferably 0.02 to 0.2 wt % and, in particular, 0.05 to 0.1 wt %.

The second essential component of the cosmetic agents according to the present invention is the menthol ester. The proportion by weight of the menthol ester to the total weight of the cosmetic agent is preferably 0.01 to 10 wt %, preferably 0.03 to 5.0 wt % and, in particular, 0.05 to 0.5 wt %.

The use of menthyl acetate has proven cosmetically especially effective, in particular in relation to preventing or reducing body odor.

The cosmetic agents exist as aqueous emulsions. Preferred cosmetic agents are characterized by a water content of 75 to 96 wt %, preferably 80 to 96 wt %, and, in particular, 85 to 94 wt % (in each case relative to the total weight of the agent).

The cosmetic agents do not include any aluminum-containing compounds. Within the scope of the present invention, the term “aluminum-containing compounds” is understood to mean antiperspirant aluminum salts and aluminum zirconium salts.

Cosmetic deodorants counteract, mask, or eliminate body odors. Body odors are produced by the action of skin bacteria on apocrine sweat, wherein unpleasant-smelling degradation products are formed. Correspondingly, preferred deodorants include active ingredients that act as germ inhibitors, enzyme inhibitors, odor absorbers, or odor maskers.

Basically, all substances that act against gram-positive bacteria are suitable as the germ inhibitors that may optionally be added to the cosmetic agents according to the present invention, examples being 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial odorants, thymol, thyme oil, eugenol, clove oil, mint oil, farnesol, phenoxyethanol, glycerol monolaurate (GML), diglycerol monocaprate (DMC), salicylic acid-N-alkylamides such as, for example, salicylic acid-n-octyl amide or salicylic acid-n-decyl amide.

Enzyme inhibitors may be added to the cosmetic agents according to the present invention, provided that they do not impair the enzyme activities that bring about the biocide activation or release in the presence of micro-organisms to be controlled. In accordance with this condition, for example, esterase inhibitors may possibly be suitable enzyme inhibitors. This preferably entails trialkyl citrates such as trimethyl citrate, tripropyl citrate, triisopropyl citrate, tributyl citrate, and, in particular, triethyl citrate (Hydagen® CAT, Henkel KGaA, Dusseldorf/FRG). The substances inhibit the enzyme activity and reduce thereby the formation of odor. Other substances that may be considered as esterase inhibitors are sterol sulfates or phosphates such as, for example, lanosterol, cholesterol, campesterol, stigmasterol and cytosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

The use of bactericides has proven especially advantageous in combination with the active ingredient combination according to the present invention. Use thereof—in particular, use of a bactericide from the group consisting of chlorhexidine, farnesol, triclosan, chloroxylenol, and phenoxyethanol, preferably phenoxyethanol had an unexpectedly advantageous effect on the odor formation by the cosmetic agents. Deodorizing cosmetic agents which include at least one bactericide from the group consisting of chlorhexidine, farnesol, triclosan, chloroxylenol, and phenoxyethanol, preferably phenoxyethanol, are therefore preferable.

The proportion by weight of the bactericide, preferably phenoxyethanol, to the total weight of the cosmetic agent is preferably 0.1 to 2.0 wt %, preferably 0.2 to 1.6 wt % and, in particular, 0.5 to 1.4 wt %.

In addition to the aforementioned active substances, the cosmetic agents may include additional active ingredients and auxiliaries. A first group of preferred cosmetic active ingredients are the oils (cosmetic oils).

The term “cosmetic oil” is understood to mean an oil that is suitable for cosmetic use and is not miscible with water. Moreover, the cosmetic oil used according to the present invention involves neither odorants nor essential oils. Deodorizing cosmetic agents that include at least one oil that is liquid at 20° C. and 1,013 hPa are preferred according to the present invention.

According to an especially preferred embodiment, the cosmetic oil that is liquid at 20° C. and 1,013 hPa is selected from the group consisting of:
(i) volatile cyclic silicone oils, in particular cyclotrisiloxane, cyclotetrasiloxane, cyclopentasiloxane, and cyclohexasiloxane, and linear silicone oils having 2 to 10 siloxane units, in particular hexamethyldisiloxane, octamethyltrisiloxane, and decamethyltetrasiloxane;
(ii) volatile non-silicone oils, in particular liquid paraffin oils and isoparaffin oils, such as isodecane, isoundecane, isododecane, isotridecane, isotetradecane, isopentadecane, isohexadecane, and isoeicosane;
(iii) non-volatile silicone oils, in particular higher molecular linear polyalkylsiloxanes;
(iv) non-volatile non-silicone oils, in particular the esters of linear or branched, saturated or unsaturated C₂₋₃₀ fatty alcohols with linear or branched, saturated or unsaturated C₂₋₃₀ fatty acids, which may be hydroxylated, the C_{8-C22} fatty alcohol esters of monohydric or polyhydric C₂-C₇ hydroxycarboxylic acids, the triethyl citrates, the branched saturated or unsaturated C₆₋₃₀ fatty alcohols, the mono-, di-, and triglycerides of linear or branched, saturated or unsaturated, optionally hydroxylated C₈₋₃₀ fatty acids, the dicarboxylic acid esters of linear or branched C₂-C₁₀ alkanols, the addition products of ethylene oxide and/or propylene oxide with monohydric or polyhydric C₃₋₂₂ alkanols, which may optionally be esterified, the symmetrical, asymmetrical, or cyclic esters of carbonic acid with fatty alcohols, the esters of dimers of unsaturated C₁₂₋₂₂ fatty acids with monohydric linear, branched, and cyclic C₂₋₁₈ alkanols or C₂₋₆ alkanols, the benzoic acid esters of linear or branched C₈₋₂₂ alkanols, such as benzoic acid C₁₂₋₁₅ alkyl esters and benzoic acid isostearyl ester and benzoic acid octyldodecyl ester, the synthetic hydrocarbons such as polyisobutene and polydecene, the alicyclic hydrocarbons; and
(v) and mixtures thereof.

The term “volatile cosmetic oil” refers to cosmetic oils that—at 20° C. and an ambient pressure of 1,013 hPa—have a vapor pressure of 2.66 Pa to 40,000 Pa (0.02 to 300 mmHg), preferably 10 to 12,000 Pa (0.1 to 90 mmHg), further preferably 13 to 3,000 Pa (0.1 to 23 mmHg) and, in particular, 15 to 500 Pa (0.1 to 4 mmHg).

The term “non-volatile cosmetic oils” is understood to mean cosmetic oils that have a vapor pressure of less than 2.66 Pa (0.02 mmHg) at 20° C. and an ambient pressure of 1,013 hPa.

Especially preferred cosmetic agents include at least one oil that is liquid at 20° C. and 1,013 hPa from the group consisting of
esters of linear or branched saturated or unsaturated C₂₋₃₀ fatty alcohols with linear or branched saturated or unsaturated C₂₋₃₀ fatty acids,
addition products of ethylene oxide and/or propylene oxide with monohydric or polyhydric C₃₋₂₂ alkanols
dialkyl ethers, and
paraffins.

These oils have proven especially effective in combination with the active ingredient combination according to the present invention made of a quaternary ammonium compound and hydroxyacetophenone.

Preferred deodorizing cosmetic agents include, relative to the total weight thereof, oil in amounts of 0.1 to 15 wt %, preferably 0.2 to 10 wt % and, in particular, 0.5 to 5.0 wt %.

Preferred cosmetic agents exist as an oil-in-water emulsion.

The agents according to the present invention include at least one emulsifier as a further preferred component. Emulsifiers that may be considered are, principally, anionic, cationic, non-ionic, and ampholytic surface-active compounds that are suitable for use on the human body. The ampholytic surface-active compounds encompass zwitterionic surface-active compounds and ampholytes. Non-ionic emulsifiers are preferred.

Non-ionic emulsifiers that can be used are, in particular, addition products of ethylene oxide with linear fatty alcohols, with fatty acids, with fatty acid alkanolamides, with fatty acid monoglycerides, with sorbitan fatty acid monoesters, with fatty acid glycerides, with methylglucoside monofatty acid esters, with polydimethylsiloxanes, and mixtures thereof.

The emulsifier is preferably selected from the group consisting of the addition products of 2 to 50 mol ethylene oxide with linear fatty alcohols having 12 to 18 carbon atoms and the addition products of 2 to 50 mol propylene oxide with linear fatty alcohols having 12 to 18 carbon atoms. Examples of especially preferred non-ionic surfactants are compounds with the INCI designations Steareth-2 and Steareth-21 and, in particular, mixtures thereof

Preferred deodorizing cosmetic agents include the non-ionic emulsifier, relative to the total weight of the agent, in an amount of 3.0 to 12 wt %, preferably 4.0 to 10 wt % and, in particular, 5.0 to 8.0 wt %.

The cosmetic agents may furthermore include at least one chelating agent, which is preferably selected from ethylenediaminetetraacetic acid (EDTA) and salts thereof as well as nitrilotriacetic acid (NTA) and mixtures of these substances, in a total amount of 0.01 to 0.5 wt %, preferably 0.02 to 0.3 wt %, in particular, 0.05 to 0.2 wt % relative to the total weight of the cosmetic agent.

As previously mentioned, the alcohol content of deodorizing cosmetic agents may be detrimental on the skin compatibility thereof. The active ingredient combination according to the present invention now makes it possible to prepare effective deodorants that have a reduced alcohol content. To improve the skin compatibility, it is therefore preferable for the deodorizing cosmetic agents to include, relative to the total weight thereof, ethanol in amounts below 5.0 wt %, preferably below 1.0 wt % and, in particular, below 0.1 wt %. It is especially preferable when the agent is free of ethanol.

In addition to the aforementioned ingredients, the deodorizing cosmetic agents may include additional active ingredients and auxiliaries. The group of these further active ingredients and auxiliaries includes, for example, the odorants.

Especially preferred cosmetic agents according to the present invention further include at least one odorant. The term “odorant” refers to a chemical compound having odor and/or taste, which excites the receptors of the hair cells of the olfactory system (adequate stimulus). The physical and chemical properties necessary for this are a low molar mass of at most 300 g/mol, a high vapor pressure, minimal water solubility and high lipid solubility, as well as weak polarity and the presence of at least one osmophoric group in the molecule In order to distinguish volatile low-molecular-weight substances that are usually (and also for purposes of the present invention) viewed and utilized not as fragrances but instead principally as solvents, for example ethanol, propanol, isopropanol, and acetone, from odorants, the odorants according to the present invention have a molar mass of 74 to 300 g/mol, include at least one osmophoric group in the molecule, and have an odor and/or taste, i.e. they excite the receptors of the hair cells of the olfactory system.

Perfumes, perfume oils, or perfume oil components may be used as odorants. Perfume oils or fragrances may, according to the present invention, be individual odorant compounds, such as synthetic products of the ester, ether, aldehyde, ketone, alcohol, and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate, and jasmecyclate. The ethers include, for example, benzyl ethyl ether and ambroxan; the aldehydes, for example, the linear alkanals having 8 to 18 carbon atoms, citral, citronellal, citronellyloxyacetaldehyde, cyclamenaldehyde, lilial, and bourgeonal; the ketones, for example, the ionones, α-isomethylionone, and methyl cedryl ketone; the alcohols, anethole, citronellol, eugenol, geraniol, linalool, phenylethyl alcohol, and terpineol; and the hydrocarbons include principally the terpenes such as limonene and pinene. It is, however, preferable to use mixtures of different odorants which together produce an appealing odorous note. Preferred cosmetic agents according to the present invention include at least one fragrance component in a total amount of 0.00001 to 10 wt %, preferably 0.5 to 5 wt %, extremely preferably 1 to 4 wt %, in each case relative to the total weight of the agent.

The compositions of some preferred cosmetic agents can be found in the following tables (all amounts given in wt % are relative to the total weight of the cosmetic agent, unless otherwise indicated).

| | Formula | Formula | Formula | Formula | Formula |
|---|---|---|---|---|---|
| | 1 | 2 | 3 | 4 | 5 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| water, optional | up to 100 | up to 100 | up to 100 | up to 100 | up to 100 |
| additives | | | | | |
| | Formula | Formula | Formula | Formula | Formula |
| | 6 | 7 | 8 | 9 | 10 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| water, optional | up to 100 | up to 100 | up to 100 | up to 100 | up to 100 |
| additives | | | | | |
| | Formula | Formula | Formula | Formula | Formula |
| | 11 | 12 | 13 | 14 | 15 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| water, optional | up to 100 | up to 100 | up to 100 | up to 100 | up to 100 |
| additives | | | | | |
| | Formula | Formula | Formula | Formula | Formula |
| | 16 | 17 | 18 | 19 | 20 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| water, optional | up to 100 | up to 100 | up to 100 | up to 100 | up to 100 |
| additives | | | | | |
| | Formula | Formula | Formula | Formula | Formula |
| | 21 | 22 | 23 | 24 | 25 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| water, optional | up to 100 | up to 100 | up to 100 | up to 100 | up to 100 |
| additives | | | | | |
| | Formula | Formula | Formula | Formula | Formula |
| | 26 | 27 | 28 | 29 | 30 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| water, optional | up to 100 | up to 100 | up to 100 | up to 100 | up to 100 |
| additives | | | | | |
| | Formula | Formula | Formula | Formula | Formula |
| | 31 | 32 | 33 | 34 | 35 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| non-ionic emulsifier | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 36 | 37 | 38 | 39 | 40 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| non-ionic emulsifier | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 41 | 42 | 43 | 44 | 45 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| non-ionic emulsifier | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 46 | 47 | 48 | 49 | 50 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| non-ionic emulsifier | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 51 | 52 | 53 | 54 | 55 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| non-ionic emulsifier* | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 56 | 57 | 58 | 59 | 60 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 61 | 62 | 63 | 64 | 65 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| non-ionic emulsifier* | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 66 | 67 | 68 | 69 | 70 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 71 | 72 | 73 | 74 | 75 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 76 | 77 | 78 | 79 | 80 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 81 | 82 | 83 | 84 | 85 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 86 | 87 | 88 | 89 | 90 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 91 | 92 | 93 | 94 | 95 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 96 | 97 | 98 | 99 | 100 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 101 | 102 | 103 | 104 | 105 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 106 | 107 | 108 | 109 | 110 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 111 | 112 | 113 | 114 | 115 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 116 | 117 | 118 | 119 | 120 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 121 | 122 | 123 | 124 | 125 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 126 | 127 | 128 | 29 | 130 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 131 | 132 | 133 | 134 | 135 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 136 | 137 | 138 | 139 | 140 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 141 | 142 | 143 | 144 | 145 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| water, optional | up to 100 | up to 100 | up to 100 | up to 100 | up to 100 |
| additives | | | | | |
| | Formula | Formula | Formula | Formula | Formula |
| | 146 | 147 | 148 | 149 | 150 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| water, optional | up to 100 | up to 100 | up to 100 | up to 100 | up to 100 |
| additives | | | | | |
| | Formula | Formula | Formula | Formula | Formula |
| | 151 | 152 | 153 | 154 | 155 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 156 | 157 | 158 | 159 | 160 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 161 | 162 | 163 | 164 | 165 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 166 | 167 | 168 | 169 | 170 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 171 | 172 | 173 | 174 | 175 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 176 | 177 | 178 | 179 | 180 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 181 | 182 | 183 | 184 | 185 |
| quaternary ammonium | 0.01 to 0.3 | 0.01 to 0.3 | 0.02 to 0.2 | 0.02 to 0.2 | 0.05 to 0.1 |
| compound | | | | | |
| enthyl acetate | 0.01 to 10 | 0.02 to 5.0 | 0.02 to 5.0 | 0.05 to 0.5 | 0.05 to 0.5 |
| non-ionic emulsifier | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 186 | 187 | 188 | 189 | 190 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| non-ionic emulsifier | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 191 | 192 | 193 | 194 | 195 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| non-ionic emulsifier | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 196 | 197 | 198 | 199 | 200 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| non-ionic emulsifier | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 201 | 202 | 203 | 204 | 205 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| non-ionic emulsifier* | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 206 | 207 | 208 | 209 | 210 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 211 | 212 | 213 | 214 | 215 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| non-ionic emulsifier* | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 216 | 217 | 218 | 219 | 220 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 221 | 222 | 223 | 224 | 225 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 226 | 227 | 228 | 229 | 230 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 231 | 232 | 233 | 234 | 235 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 236 | 237 | 238 | 239 | 240 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 241 | 242 | 243 | 244 | 245 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 246 | 247 | 248 | 249 | 250 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 251 | 252 | 253 | 254 | 255 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifie* | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 256 | 257 | 258 | 259 | 260 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| bactericide | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 261 | 262 | 263 | 264 | 265 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 266 | 267 | 268 | 269 | 270 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| liquid oil (20° C., | 0.1 to 15 | 0.2 to 10 | 0.2 to 10 | 0.5 to 5.0 | 0.5 to 5.0 |
| 1,013 hPa) | | | | | |
| non-ionic emulsifier | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 271 | 272 | 273 | 274 | 275 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 276 | 277 | 278 | 279 | 280 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| liquid oil (20° C., | 0.1 to 15 | 0.2 to 10 | 0.2 to 10 | 0.5 to 5.0 | 0.5 to 5.0 |
| 1,013 hPa) | | | | | |
| non-ionic emulsifier | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 281 | 282 | 283 | 284 | 285 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 286 | 287 | 288 | 289 | 290 |
| quaternary ammonium | 0.01 to 0.3 | 0.01 to 0.3 | 0.02 to 0.2 | 0.02 to 0.2 | 0.05 to 0.1 |
| compound | | | | | |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| ethanol | <5.0 | <1.0 | <1.0 | <1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 291 | 292 | 293 | 294 | 295 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| ethanol | <5.0 | <1.0 | <1.0 | < 1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |
| | Formula | Formula | Formula | Formula | Formula |
| | 296 | 297 | 298 | 299 | 300 |
| benzalkonium chloride | 0.01 to 0.3 | 0.01 to 0.3 | 0.02 to 0.2 | 0.02 to 0.2 | 0.05 to 0.1 |
| menthyl acetate | 0.01 to 10 | 0.02 to 5.0 | 0.02 to 5.0 | 0.05 to 0.5 | 0.05 to 0.5 |
| phenoxyethanol | 0.1 to 2.0 | 0.1 to 2.0 | 0.2 to 1.6 | 0.2 to 1.6 | 0.5 to 1.4 |
| non-ionic emulsifier* | 3.0 to 12 | 3.0 to 12 | 4.0 to 10 | 4.0 to 10 | 5.0 to 8.0 |
| liquid oil (20° C., | 0.1 to 15 | 0.2 to 10 | 0.2 to 10 | 0.5 to 5.0 | 0.5 to 5.0 |
| 1,013 hPa) | | | | | |
| ethanol | <5.0 | <1.0 | <1.0 | < 1.0 | <0.1 |
| Water | 75 to 96 | 80 to 96 | 85 to 94 | 85 to 94 | 85 to 94 |
| optional additives | up to 100 | up to 100 | up to 100 | up to 100 | up to 100 |

*Steareth-2, Steareth-21, or combinations thereof

The deodorizing cosmetic agent may be applied by means of different methods. According to a preferred embodiment, the antiperspirant cosmetic agent is prepared as a spray application. The spray application is performed with a spray device containing a container filled with the deodorizing cosmetic agent. The filling may be under the pressure of a propellant (pressure gas can, pressure gas packaging, aerosol packaging), or may entail a pump sprayer that is used mechanically, without propellant gas (pump spray/squeeze bottle). It is preferable to forgo a propellant gas, and therefore preferred agents are propellant-free. The resulting preferred propellant-free deodorizing agents are preferably applied by means of a pump spray. A pump spray containing a deodorizing cosmetic agent according to the present invention is therefore another subject matter of the present invention.

The deodorizing agent may thus furthermore be manufactured as a roll-on. Such manufacturing is especially preferred, in addition to manufacturing as a pump spray. The application may take place with, for example a roller ball applicator. Such rollers have a ball bearing, mounted within a ball bed, which can be moved by motion over a surface. Then, the ball takes up some of the antiperspirant cosmetic agent according to the present invention to be distributed and transports same to the surface to be treated. Roll-on applicators containing a deodorizing cosmetic agent according to the present invention are preferred.

A suitable alternative form of application of the deodorizing cosmetic agent is combination with a disposable substrate, e.g., a substrate selected from the group consisting of cloths, pads, and pledgets. Particularly preferred are moist wipes, i.e., moist wipes which are prepackaged, preferably individually packaged, for the user, such as are well known, for example, from the area of glass cleaning or from the area of moist toilet wipes. Such moist wipes, which advantageously may also include preservatives, are impregnated or loaded with a deodorizing cosmetic agent according to the present invention and preferably individually packaged. They may be used, for example, as a deodorant wipe, which is of particular interest for usage while out and about. Preferred substrate materials are selected from porous flat wipes. They may be composed of a fibrous or cellular flexible material that has sufficient mechanical stability and, at the same time, softness for use on skin. These wipes include wipes made of woven and nonwoven (fleece) synthetic and natural fibers, felt, paper, or foam, such as hydrophilic polyurethane foam. According to the present invention, preferred deodorizing or antiperspirant substrates may be obtained by soaking or impregnating, or also melting, an antiperspirant cosmetic agent according to the present invention on a substrate.

Another subject matter being claimed is a non-therapeutic cosmetic method for preventing and/or reducing body odor, in which the cosmetic agent according to the present invention is applied to the skin, in particular, to the skin of the armpits, and left on the skin for at least 1 hour, preferably for at least 2 hours, preferably for at least 4 hours, in particular, for at least 6 hours.

As previously mentioned, the cosmetic agents according to the present invention have an advantageous effect on the formation of body odor. The use of these cosmetic agents to prevent and/or reduce body odor is another subject matter of the present invention.

### EXAMPLES

The following deodorant emulsions were produced (amounts given in wt %).

| | 1 | 2 | 3 | 4 | 5 | 6 |
|---|---|---|---|---|---|---|
| PPG-15 Stearyl | 3.0 | 3.0 | 3.0 | 3.0 | 3.0 | 3.0 |
| Ether | | | | | | |
| Steareth-2 | 2.5 | 2.5 | 2.5 | 2.5 | 2.5 | 2.5 |
| Steareth-21 | 1.8 | 1.8 | 1.8 | 1.8 | 1.8 | 1.8 |
| Menthyl acetate | 0.085 | 0.085 | 0.76 | — | 0.76 | — |
| Barquat¹⁾ | 0.2 | 0.2 | 0.1 | 0.2 | — | — |
| Triethyl citrate | — | — | — | — | — | 2.0 |
| Phenoxyethanol | 0.9 | 0.9 | 0.9 | 0.9 | 0.9 | 0.9 |
| 1,2-Propanediol | 3.0 | 3.0 | 3.0 | 3.0 | 3.0 | 3.0 |
| EDTA | 0.1 | 0.1 | 0.1 | 0.1 | 0.1 | 0.1 |
| Perfume | 1.0 | 1.0 | 1.0 | 1.0 | 1.0 | 1.0 |
| Water | up to | up to | up to | up to | up to | up to 100 |
| | 100 | 100 | 100 | 100 | 100 | |
| Stability | stable | stable | stable | stable | stable | Phase |
| (4 weeks, 40° C.) | | | | | | separation |
| Effect against | + | + | + | − | − | − |
| Body odor | | | | | | |

¹⁾INCI: Benzalkonium chloride, 50% (Lonza)

The compositions 1 to 3 according to the present invention are characterized by high storage stability, high microbiological stability, and a favorable action against body odor.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A deodorizing cosmetic agent in the form of an aqueous emulsion, comprising:
at least one quaternary ammonium compound, and
at least one menthol ester,
wherein the deodorizing cosmetic agent does not include any aluminum-containing compounds.

2. The deodorizing cosmetic agent according to claim 1, wherein the at least one quaternary ammonium compound includes a compound selected from the group consisting of: behentrimonium chloride, cetrimonium bromide, cetrimonium chloride, laurtrimonium bromide, laurtrimonium chloride, steartrimonium bromide, steartrimonium chloride, benzethonium chloride, benzalkonium chloride, and mixtures thereof.

3. The deodorizing cosmetic agent according claim 1, wherein the at least one quaternary ammonium compound includes benzalkonium chloride.

4. The deodorizing cosmetic agent according to claim 1, wherein the quaternary ammonium compound is included in an amount, relative to the total weight of the agent, of 0.01 to 0.3 wt %.

5. The deodorizing cosmetic agent according to claim 1, wherein the quaternary ammonium compound is included in an amount, relative to the total weight of the agent, of 0.02 to 0.2 wt %.

6. The deodorizing cosmetic agent according to claim 1, wherein the quaternary ammonium compound is included in an amount, relative to the total weight of the agent, of 0.05 to 0.1 wt %.

7. The deodorizing cosmetic agent according to claim 1, wherein the at least one menthol ester includes menthyl acetate.

8. The deodorizing cosmetic agent according to claim 1, wherein the at least one menthol ester is included in an amount, relative to the total weight of the agent, of 0.01 to 10 wt %.

9. The deodorizing cosmetic agent according to claim 1, wherein the at least one menthol ester is included in an amount, relative to the total weight of the agent, of 0.03 to 5.0 wt %.

10. The deodorizing cosmetic agent according to claim 1, wherein the at least one menthol ester is included in an amount, relative to the total weight of the agent, of 0.05 to 0.5 wt %.

11. The deodorizing cosmetic agent according to claim 1, wherein, relative to the total weight of the agent, ethanol is included in amounts below 5.0 wt %.

12. The deodorizing cosmetic agent according to claim 1, wherein, relative to the total weight of the agent, ethanol is included in amounts below 1.0 wt %.

13. The deodorizing cosmetic agent according to claim 1, wherein, relative to the total weight of the agent, ethanol is included in amounts below 0.1 wt %.

14. The deodorizing cosmetic agent according to claim 1, wherein, relative to the total weight of the agent, water is included in amounts of 75 to 96 wt %.

15. The deodorizing cosmetic agent according to claim 1, wherein, relative to the total weight of the agent, water is included in amounts of 80 to 96 wt %.

16. The deodorizing cosmetic agent according to claim 1, wherein, relative to the total weight of the agent, water is included in amounts of 85 to 95 wt %.

17. A non-therapeutic cosmetic method for preventing and/or reducing body odor, comprising applying to skin the deodorizing cosmetic agent according to claim 1, and leaving the deodorizing cosmetic agent on the skin for at least 1 hour.
